(19) United States
(12) Patent Application Publication
(10) Pub. No.: US 2013/0150970 A1
(43) Pub. Date: Jun. 13, 2013

(54) Implant with Sensor

(71) Applicant: Basix Spine LLC, Irvine, CA (US)

(72) Inventor: Gowriharan Thaiyananthan, Whittier, CA (US)

(73) Assignee: BASIX SPINE LLC, Irvine, CA (US)

(21) Appl. No.: 13/683,105

(22) Filed: Nov. 21, 2012

Related U.S. Application Data
(60) Provisional application No. 61/562,268, filed on Nov. 21, 2011.

(51) Int. Cl. A61F 2/44

(52) U.S. Cl. CPC A61F 2/442
USPC 623/17.16

(57) ABSTRACT

A spinal implant for supporting adjacent bones or vertebrae by insertion through a surgical opening with adjacent nerves is disclosed. The spinal implant may include a body and one or more conductors. The body is configured to be inserted through the surgical opening to fit between the two endplates of the adjacent vertebrae and to maintain spacing between the endplates. The conductor has at least a first and second ends. Extending through a portion of the body, the conductor's first end is exposed and accessible through the surgical opening. The second end is exposed and positioned to communicate with at least one of the nerves. The first end of the conductor is also configured to communicate with a monitori

### CROSS-REFERENCE TO RELATED APPLICATIONS

This application claims priority to U.S. Provisional Application No. 61/562,268, filed Nov. 21, 2011, which is herein incorporated in its entirety by reference.

### BACKGROUND

Bone fusion surgery often requires navigation through delicate, nervated tissue structures for placement of an implant. For example, spine fusion surgery may include placing a cage or body into a space between a pair of adjacent vertebrae, i.e., into the intervertebral space, so as to maintain spacing between the vertebrae and facilitate fusion. Improper placement or positioning of the implant can aggravate spine pain by impinging on nerves extending along or adjacent the intervertebral space.

It is desirable to improve surgical tools and methods for bone fusion so as to avoid impingement on nerves and other proximal or affected tissue structures and provide an anterior or posterior spinal fusion through the axia of the nerve roots.

### SUMMARY

Implementations of the present disclosure overcome the problems of the prior art by providing a spinal implant for supporting adjacent bones or vertebrae by insertion through a surgical opening with adjacent nerves. The spinal implant may include a body and one or more conductors. The body is configured to be inserted through the surgical opening, whether anterior cervical, lumbar, lumbar lateral, posterior lumbar, or oblique lumbar, to fit between the two endplates of the adjacent vertebrae or in a pedicle, and to maintain spacing between the endplates. The conductor has at least a first and second end. Extending through a portion of the body, the conductor's first end is exposed and accessible through the surgical opening. The second end is exposed and positioned to communicate with at least one of the nerves. The first end of the conductor is also configured to communicate with a monitoring device.

The conductor may include a draw rod and a distal tip, wherein the distal tip is configured for attachment to a distal end of the body. A proximal end of the draw rod may be the first end of the conductor and a distal end may contact a proximal end of the tip. A distal end of the tip may be the second end of the conductor. The tip may taper from the proximal end to the distal end to ease insertion or for other purposes.

The conductor may also include one or more relays. Each of the relays has a first end configured to establish communication with the draw rod and a second end exposed to one of the nerves.

A top and bottom surfaces of the implant body may include gripping structure to facilitate adhesion to the adjacent bony structure.

Also, the body may define an opening extending between the top and bottom surfaces. Other openings may be defined between lateral surfaces of the body. Such openings may have advantages such as providing for a lighter weight or space for packing with bone graft or growth promotion material.

An axial opening may be defined through the body to allow passage of the draw rod therethrough. If the body includes the opening between the top and bottom surfaces, the draw rod may also extend transversely through the opening. The axial opening may be threaded for mating with threads of the draw rod.

The body may also have a concave lateral surface and a convex lateral surface to form a curved shape for use in transforaminal lumbar interbody fusion, for example. The conductor may be a central rod that has a first end and a second end. The first end of the rod may extend through a first end of the body and the second end of the rod through the second end of the body. Additionally, relays may be included extending through the convex and concave lateral surfaces and coupled in communication with the central rod.

The body may also have an anterior flat surface and a posterior curved surface for use in cervical fusion. The conductor may include a central rod extending through the anterior and posterior surfaces anchored cage/stand-alone/no-profile/zero-profile cages referenced, with screws and/or blade/shim/deployable spike fixation. In a further example, the body may include a plurality of screws for attaching the implant to adjacent vertebra

A method of using an implant may include inserting a body of the implant through a surgical opening into a space between two bones. An electrical field from adjacent nerves is sensed with at least one conductor extending through a portion of the body. Adjustments are made to positioning of the implant based on the sensing of the electrical field.

These and other features and advantages of the implementations of the present disclosure will become more readily apparent to those skilled in the art upon consideration of the following detailed description and accompanying drawings, which describe both the preferred and alternative implementations of the present disclosure.

### BRIEF DESCRIPTION OF THE DRAWINGS

FIG. 1 is a perspective view of a spine implant including a cage or body and a plurality of conductors;

FIG. 2 is an exploded view of FIG. 1;

FIG. 3 is a schematic of an implant position sensing system using, for example, the implant of FIGS. 1 and 2;

FIG. 4 is another implant with a curved shape used for a TLIF procedure;

FIG. 5 is another implant with a rounded posterior surface and a central draw rod conductor;

FIG. 6 is another implant with a rectangular body and laterally positioned relay conductors;

FIG. 7 is another implant with a curved posterior surface and internal relays extending therethrough;

FIG. 8 shows an access approach for a PLIF procedure used for a spine implant and a position of adjacent nerves;

FIG. 9 shows an access approach for an ALIF procedure used for a spine implant;

FIG. 10 shows an access approach for a TLIF procedure used for a spine implant;

FIG. 11 shows an access approach for a lateral lumbar interbody fusion procedure used for a spine implant;

FIG. 12 shows another implant with an attachment feature; and

FIG. 13 shows an access approach for a procedure using an implant including an attachment feature.

### DETAILED DESCRIPTION

Implementations of the present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

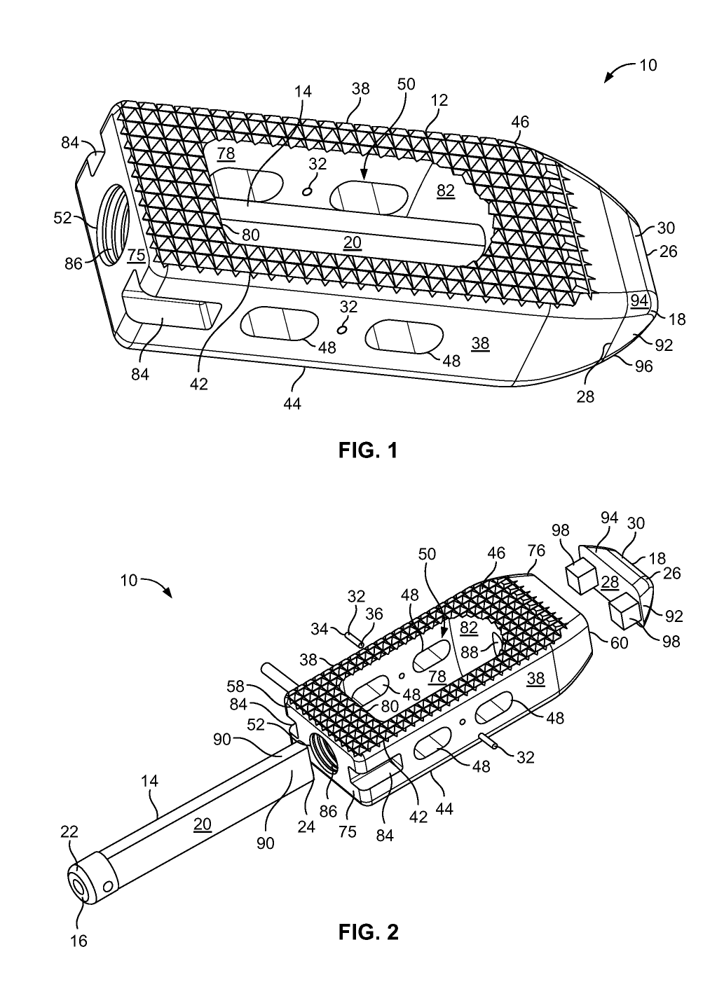

As shown for example in FIGS. 1 and 2, a spine implant 10 is disclosed that includes a body 12 and one or more conductors. The spine implant 10 is inserted through a surgical opening to fit between two endplates of adjacent vertebrae and maintain their spacing. One of the conductors 14 has a first end 16 and a second end 18 and extends through a portion of the body 12. The first end 16 is exposed and accessible through the surgical opening and may be configured for coupling with and communicating signals to a monitoring device. The second end 18 is exposed out of the body 12 and positioned to communicate with one or more nerves within the patient.

Advantageously, the spine implant 10 and its one or more conductors 14 integrated into the body 12 provide numerous platforms for the direct monitoring of neurological elements as well as guidance during implanting. Such guidance can be had without using fluoroscopy to determine placement. The spine implant 10 can be customized to many different surgical approaches, such as ALIF, TLIF, PLIF, or lateral lumbar interbody fusion.

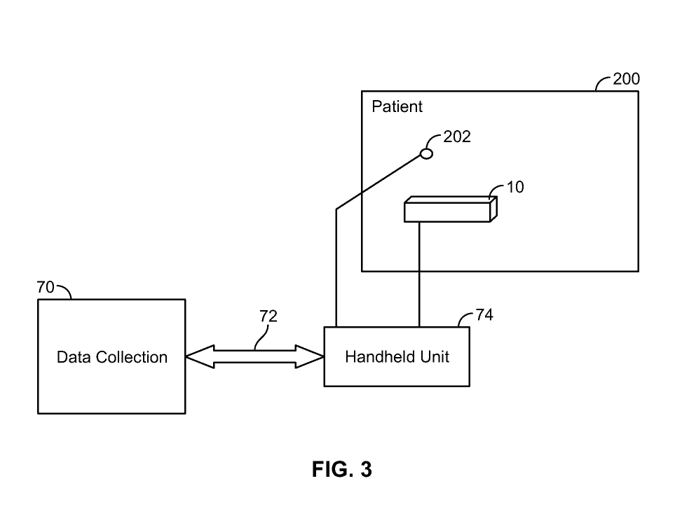

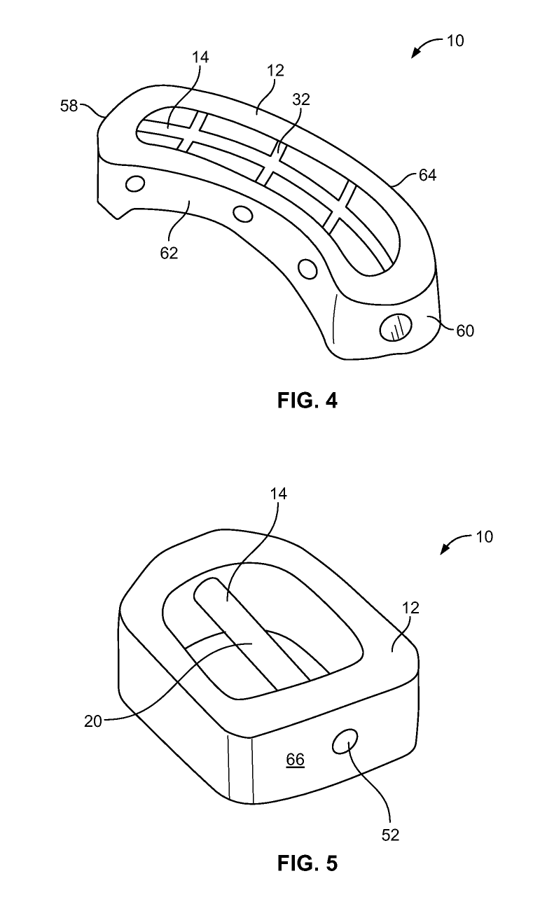

As shown in FIG. 3, as part of an implant and monitoring system, the spine implant 10 may be connected to a handheld unit 74 which, in turn, is connected by communication lines 72 (or wirelessly) to a data collection computer system 70, such as an iPad®, laptop or desktop computer. The computer system 70, handheld unit 74 or other various hardware, firmware and/or software combinations make up a monitoring unit. The handheld unit 74 preferably has a power source and controls that enable it to send a stimulation signal through the spine implant. Also, a return line from one or more EMG (electromyogram) electrodes 202 connected to the handheld unit 74 allow it to measure the response of the surrounding tissues of a patient 200 stimulated by the conductors 14. The handheld unit 74 may have its own processing power that enables it to receive and process the data signal from the implant 10, or it may transmit relatively more raw data to the computer system 70 over communication lines 72 for further processing.

During use, the handheld unit 74 stimulates the spine implant with an electrical current. The conductors 14 are placed along muscle groups in the patient to stimulate EMG potential from muscle groups enervated by specific nerve roots. Software resident on the handheld unit 74 or data collection computer system 70 is configured to assess the current passed through the graft. The software is also configured to interpret the EMG signals to determine if, at a particular threshold, there is an appreciable change in the EMG pattern which would indicate that the implant is close to a particular neural tissue of interest. For example, the EMG threshold may range from −2000 to 2000. The signal, root, or a neuromodulation based system may be displayed on the computer system 70 using a color scheme to indicate passage of various thresholds, such as green, yellow and red to provide intra-operative feedback to the surgeon indicating proximity to a particular nerve root.

Although the handheld unit 74 and computer system 70 are described herein, the spine implant 10 could be employed with conventional stimulation and sensing systems as long as some source of electrical stimulation and/or some system for determining the return signals is supplied.

Returning now to FIGS. 1 and 2, the body 12 of the spine implant 10 has a generally rectangular block shape with a pair of opposed lateral surfaces 38, a top surface 42, a bottom surface 44, a proximal surface 75 and a distal surface 76. The opposed lateral surfaces 38 and top and bottom surfaces 42, 44, taper towards each other as they extend toward the distal surface 76. Generally, this taper begins at approximately the final 10% to 20% of the length of the body 12. The taper may be configured to smoothly transition to a proximal end 28 of a tip 26 of the conductor 14. As will be described below, the tip 26 of the conductor 14 also continues the taper in the distal direction to facilitate insertion into the surgically prepared intervertebral opening.

The top and bottom surfaces 42, 44 and the opposed lateral surfaces 38 have a length that is longer than either the height of the opposed lateral surfaces 38 and/or the width of the top and bottom surfaces 42, 44. This length allows insertion of the implant 10 into the low height but relatively long intervertebral space. A width of the implant at the proximal surface 75 and the distal surface 76 is less than the length of the body 12 but greater than the height of the body 12.

It should be noted that these dimensions are exemplary and may be varied (as shown by further examples hereinbelow) depending upon the surgical access point(s) and the bones being supported or fused. Non-spinal applications are also possible (in which case different dimensions of the implant will be used) wherein bones or other joints are connected or fused, such as knee or elbow joints, and avoidance of nerve, muscle or other tissues is desired.

Each of the top and bottom surfaces 42, 44 may define a gripping structure 46, such as a plurality of small pyramidal structures. The gripping structure 46 is configured to grip and promote fixation and eventual bony in-growth of the upper and lower endplates of the adjacent vertebrae. Other gripping structures 46 may be employed, including structures of varying roughness, microstructure or macrostructure, and texture customized to the desired surgical objectives and the anatomy of the joint being fused. The gripping structure 46 may terminate or transition to a smooth surface as the top and bottom surfaces 42, 44 begin to taper toward each other and the conductor 14.

Defined in the body 12 is a plurality of openings to contain bone graft or growth materials and promote bony in-growth for fusion of the adjacent vertebrae. For example, the body 12 of FIGS. 1 and 2 includes a vertical opening 50 extending between the top and bottom surfaces 42, 44. The vertical opening 50 has a roughly rectangular shape with a pair of flat, laterally facing sides 78 and a flat proximal side 80, but with the exception of a curved distal side 82. Generally, the size of the opening facilitates communication between the bony end-plates, any graft or in-growth promoting material (such as bone morphogenic protein) therein, and eventual permanent fusion.

Also defined in the body 12 may be a plurality of lateral openings 48, and in particular four openings, two each extending between one of the lateral sides 78 of the vertical opening 50 and the adjacent one of the opposed lateral surfaces 38 of the body 12. The lateral openings 48 have channel shapes with curved ends and, similar to the vertical opening 50, can facilitate bony in-growth through access to bone graft or growth materials.

Defined on the distal lateral end of the body 12 is a pair of laterally spaced notches 84. Each of the notches 84 can extend through a respective one of the corners defined by the distal and lateral surfaces 76 of the body 12. The notches 84 may be configured to hold bone graft or growth material during insertion of the body 12. The notches 84 may also be configured to prevent the distal end of the body 12 from rotating.

Extending along the length of the body, through and between the proximal surface 75 and the distal surface 76, is an axial opening 52 that has a proximal portion 86 and a distal portion 88. The proximal portion 86 is defined between the proximal side 80 of the vertical opening 50 and the proximal surface 75 of the body 12. The distal portion 88 is defined between the distal side 82 of the vertical opening 50 and the distal surface 76 of the body 12. The axial opening 52 may have a cylindrical shape and include threads in the proximal and distal portions 86, 88.

The body could be comprised of titanium, carbon, hybrid composites, PEEK, an allograft, and/or a combination of materials.

As shown in FIG. 2, the conductor 14 includes a draw rod 20 and a tip 26. The draw rod 20 includes a proximal end 22 (which may be threaded) and a distal end 24. The proximal end 22 may have a cylindrical portion that is configured to slide into the proximal portion 86 of the cylindrically shaped axial opening 52. For its distal end 24, the draw rod 20 has a pair of lateral side surfaces 90 formed in the cylindrical cross-section. These lateral side surfaces 90 mate with flat surfaces on the lateral sides of the distal portion 88 of the axial opening 52. The flat lateral side surfaces 90 thus become an anti-rotation feature and facilitate good contact with the relays 32.

As shown in FIG. 1, when in an assembled condition, the draw rod 20 has the flat lateral side surfaces 90 positioned laterally and against flat surfaces of the distal portion 88. Also, the proximal end 22 of the draw rod 20 is recessed within the proximal portion 86 of the axial opening 52, leaving threads within the proximal portion exposed.

Referring again to FIG. 2, the tip 26 of the conductor 14 has the proximal end 28 and a distal end 30. The tip 26 may taper from the proximal end 28 to the distal end 30. Similar to the body 12, the tip 26 may include a pair of lateral surfaces 92 that taper toward each other and a top surface 94 and a bottom surface 96 that taper toward each other. At its distal-most end, the tip 26 may be rounded or chamfered for a soft, a-traumatic contour.

The tip 26 also includes a spaced pair of mounting structures 98 that extend proximally from a flat surface of the proximal end 28. The mounting structures 98, for example, have square block shapes and are configured to mate with congruent openings defined in the distal, second end surface 60 of the body 12. The mounting structures 98 could have other shapes, such as peg, rectangular or irregular shapes that facilitate secure attachment of the tip 26 to the body 12.

The conductors of the spine implant 10 include relays 32 that include a first end 34 and a second end 36. For example, FIGS. 1 and 2 show three relays 32. Two of the relays extend laterally away from the lateral side surfaces 90 of the draw rod 20 through openings in the opposed lateral surfaces 38 of body 12. One of the relays 32 is positioned more proximally and extends laterally through the body 12 into one of the notches 84 (not shown). In the assembled configuration, each of the relays 32 has the first end 34 positioned in communication contact with the draw rod 20 and the second end 36 positioned to contact one or more selected nerves (such as the spinal cord and its branch nerves) for stimulation.

As shown in FIG. 1, the fully assembled spine implant 10 has the tip 26 mounted to the body 12 via the mounting structures 98 and the proximal end 28 of the tip 26 abuts the distal end 24 of the draw rod 20. The draw rod 20 extends through both the proximal portion 86 and distal portion 88 of the axial opening 52. The middle of the draw rod 20 extends through the vertical opening 50. The relays 32 each have the first end 34 in abutting contact with the lateral side surfaces 90 of the draw rod 20 and a second end 36 extending through respective openings in the body 12 for exposed contact with surrounding tissues. The interconnection of the draw rod 20 and the relays 32 establishes electrical communication with the surrounding muscle, nerve and other tissue structures.

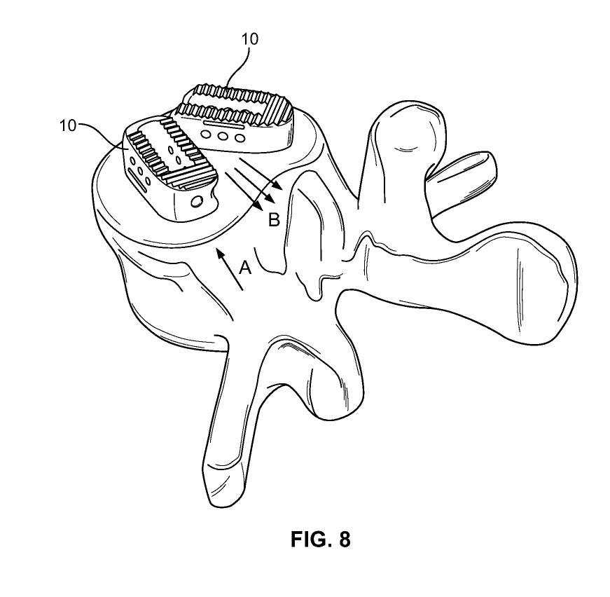

The spine implant 10 may have different shapes or sizes of body 12 and configurations of conductors 14 depending upon the surgical approach, the joint being fused, and the surrounding tissue configuration. The spine implant 10 of FIGS. 1 and 2 may be employed, for example, in a posterior lumbar interbody fusion (PLIF) procedure wherein two of the implants are used, one each inserted laterally adjacent to the spinal cord. The PLIF procedure includes resection of the lamina and part of the facets to provide access for the spine implant 10 from the posterior spine. The nerves are refracted and the disc removed and then the implants 10 are inserted directly or straight into the surgical opening with the patient in a prone position. FIG. 8 shows a direction with arrow (A) of insertion of the two of the spine implants 10. The direction of the spinal cord and sensing field is illustrated with arrows (B). Monitoring of the signals from the spine implant 10 during this procedure helps to avoid injury to the nerve structures.

FIG. 4 shows another spine implant 10 wherein the body 12 has a curved shape with a concave lateral surface 62 and convex lateral surface 64 extending between a first end surface 58 and a second end surface 60. One of the conductors 14 is a main rod extending in a curved arc between and through the end surfaces 58, 60. And, relays 32 have ends extending between and through the concave and convex lateral surfaces 62, 64.

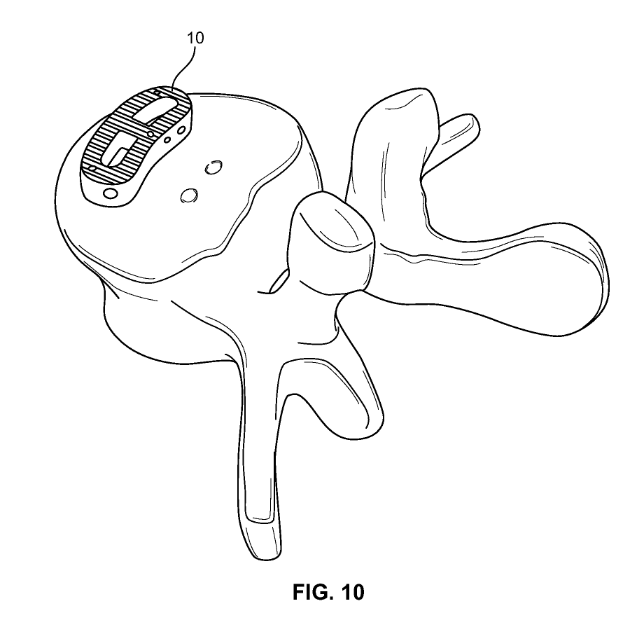

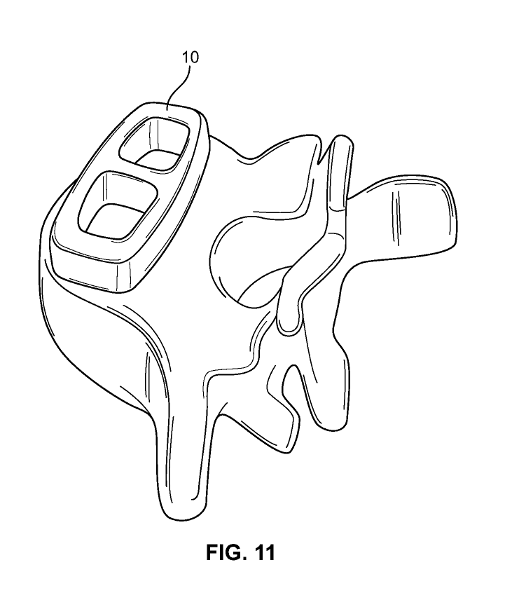

The curved shape of the body 12 of FIG. 4 is configured for transforaminal lumbar interbody fusion (TLIF) wherein the full facets are removed to give a wide berth to the spinal cord. In the TLIF procedure, the patient is positioned prone, the facet is removed, the nerves retracted, and the disc removed. The curved shape of the body 12 facilitates the rotating-sliding (pseudo-lateral) insertion through the TLIF port along the anterior arc of the vertebral endplates, as shown in FIG. 10.

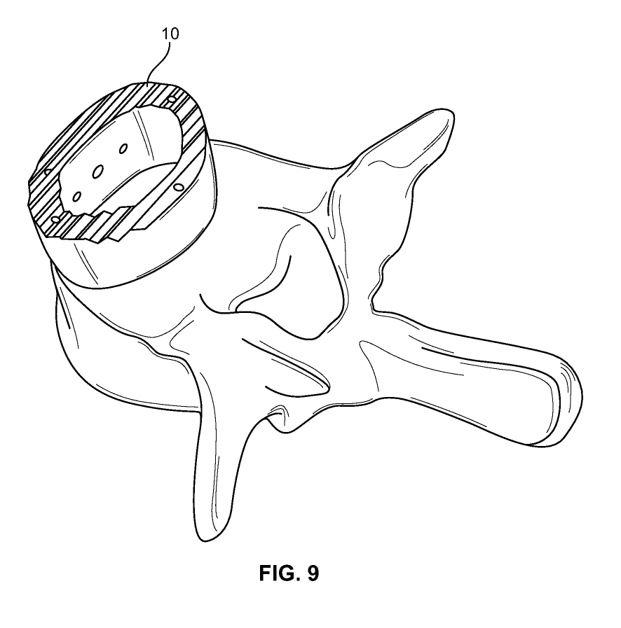

FIG. 5 shows another spine implant 10 configured for an anterior lumbar interbody fusion (ALIF). A single draw rod 20 of conductor 14 may be used in an example ALIF-type implant 10. The implant includes an anterior flat surface 66 and a posterior curved surface 68 (not shown). During an ALIF procedure, the patient is positioned supine, the disc is removed from anterior access and the implant is inserted straight or direct from the anterior position. As shown in FIG. 9, in the ALIF procedure the body 12 of the implant 10 is shaped and configured to nearly fill the entire intervertebral space between the endplates.

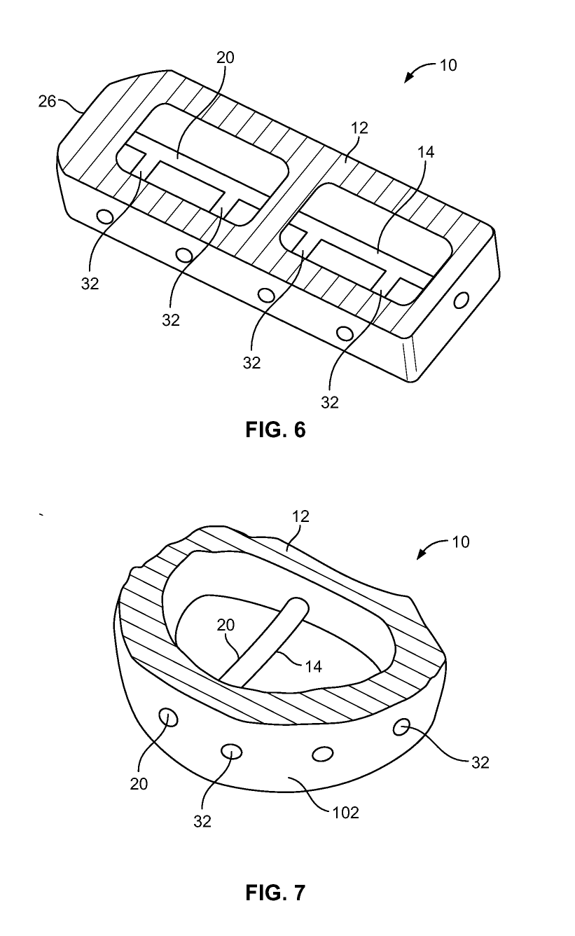

FIG. 6 shows another spine implant 10 fashioned for PLIF or lateral lumbar interbody fusion uses, including the tapered tip 26 and the draw rod 20, but with multiple relays 32 extending laterally to one side. For example, the implant 10 can include four relays 32 extending from one of the side of the implant 10. The side with the relays 32 may be oriented to face laterally toward the tissue on the sides likely to have nerves, while the other lateral edge faces toward the center of the intervertebral space where no nerves are present.

FIG. 7 shows another spine implant 10 with the body 12 configured for use in an ALIF procedure that includes an anterior flat surface 100 (not show) and a posterior curved surface 102. The posterior curved surface 102 has exposed thereon a plurality of the relays 32 that are connected in communication with the draw rod 20 by conductors within the wall structure of the body 12.

It should be noted that the examples of different surgical procedures and shapes of the body 12 and configurations of the conductors 14 need not be limited to those shown herein. Other adaptations are possible, or the implants shown herein may be used, for different procedures such as an extreme lateral interbody fusion procedure. In the procedure, the patient is positioned on their side and access is vertical through the lateral edge of the intervertebral disc space (as shown in FIG. 11) to avoid the anterior nerves and posterior bony structures.

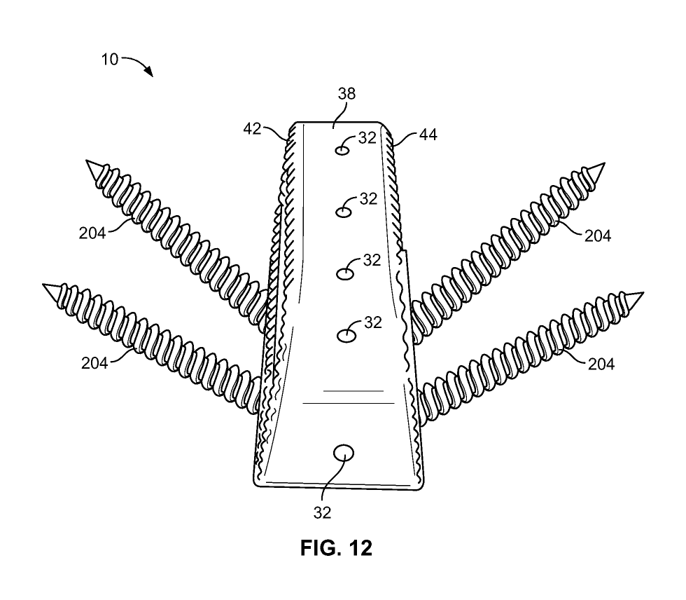

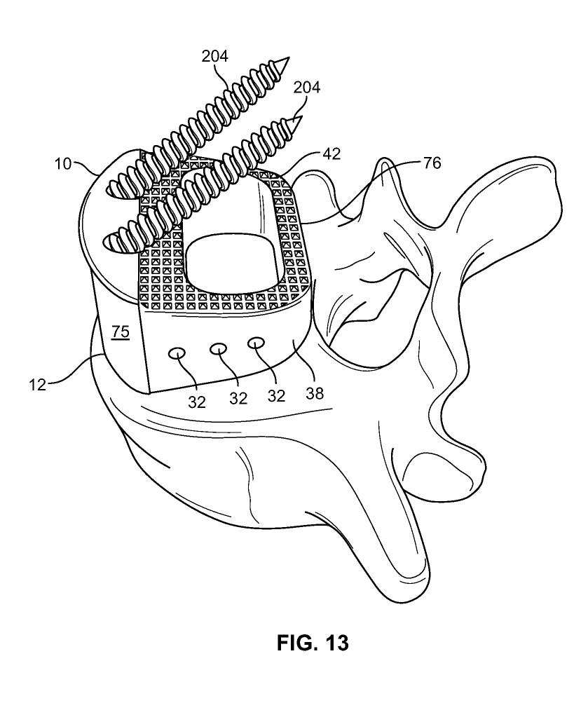

FIGS. 12 and 13 show another spine implant 10 including an attachment feature 204 for joining the implant 10 to adjacent vertebra. For example, the attachment feature 204 can include threaded screws extending from the top surface 42 and the bottom surface 44 of the body 12 of the implant 10. The opposed lateral surfaces 38 and the distal surface 76 can include a plurality of relays 32 that are connected in communication with the draw rod 20 by conductors within the wall structure of the body 12. Each of the relays 32 can be individually monitored to determine the current at each of the individual contact points of the relays 32 around the body 12. As illustrated in FIGS. 12 and 13, the attachment feature 204 can be inserted and/or manipulated via an access point on the proximal surface 75 of the body 12. The attachment feature 204 can be configured to enter the adjacent vertebral body at an angle.

During placement of the spine implant 10, the draw rod 20 is threaded into the axial opening 52 and the relays 32 placed in their respective openings. An inserter is threaded into the threaded proximal portion 86 of the axial opening 52. This inserter is part of, or connected to, the handheld unit 74 to establish communication between the implant 10 and the handheld unit. Also, the inserter serves as handle for insertion of the implant 10. The implant 10 is then placed in the disc space and the transfer of data from the relays 32 begins. Real-time data on the EMG responses will be displayed on the screen of the computer system 70 to guide the surgeon's positioning of the implant 10.

Any combination of one or more computer readable medium(s) may be utilized, such as for the computer system 70 of FIG. 3. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present system (such as the system shown in FIG. 3) may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present system are described above with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A number of aspects of the systems, devices and methods have been described. Nevertheless, it will be understood that various modifications may be made without departing from the spirit and scope of the disclosure. Accordingly, other aspects are within the scope of the following claims.

That which is claimed:

1. A spinal implant for supporting adjacent vertebrae by insertion through a surgical opening with adjacent nerves, the spinal implant comprising:
a body configured to be inserted through the surgical opening to fit between two endplates of the adjacent vertebrae and to maintain spacing between the endplates; and
at least one conductor having at least a first and second ends, the conductor extending through a portion of the body wherein the first end is exposed and accessible through the surgical opening and a second end exposed and positioned to communicate with at least one of the nerves;
wherein the first end is configured for coupling in communication with a monitoring device.

2. A spinal implant of claim 1, wherein the at least one conductor includes a draw rod.

3. A spinal implant of claim 2, wherein the at least one conductor includes a tip configured for attachment to a distal end of the body.

4. A spinal implant of claim 3, wherein the draw rod includes a proximal end that is the first end and a distal end that contacts a proximal end of the tip and a distal end of the tip is the second end.

5. A spinal implant of claim 4, wherein the at least one conductor includes at least one relay.

6. A spinal implant of claim 5, wherein the at least one relay includes a first end configured to establish communication with the draw rod and a second end exposed to one of the nerves.

7. A spinal implant of claim 6, wherein the tip tapers from the proximal end to the distal end.

8. A spinal implant of claim 7, wherein an outer surface of the tip has a smooth transition to an adjacent outer surface of the body.

9. A spinal implant of claim 8, wherein the tip and body have a pair of opposing lateral surfaces and a pair of opposing top and bottom surfaces that include taper in the distal direction to the distal end of the tip.

10. A spine implant of claim 9, wherein the top and bottom surfaces of the body have gripping structure.

11. A spine implant of claim 10, wherein the lateral surfaces of the body define openings extending through the body.

12. A spine implant of claim 11, wherein the top and bottom surfaces of the body define an opening extending therebetween.

13. A spine implant of claim 12, wherein the body defines an axial opening through which the draw rod is configured to extend and wherein the draw rod extends through the opening extending between the top and bottom surfaces.

14. A spine implant of claim 13, wherein the axial opening includes a threaded portion configured to mate with a threaded portion of the draw rod.

15. A spine implant of claim 1, wherein the body has a first end surface, a second end surface, a concave lateral surface, a convex lateral surface, a top surface and a bottom surface.

16. A spine implant of claim 15, wherein the at least one conductor includes a central rod including the first and second ends, wherein the first end extends through the first end of the body and the second end extends through the second end of the body.

17. A spine implant of claim 16, wherein the at least one conductor includes at least one relay extending through the convex and concave lateral surfaces and coupled in communication with the central rod.

18. A spine implant of claim 17, wherein the body defines an opening extending between the top and bottom surfaces.

19. A spine implant of claim 1, wherein the body includes an anterior flat surface and a posterior curved surface and wherein the at least one conductor includes a central rod extending through the anterior flat surface at the first end and the posterior curved surface at the second end.

20. A spine implant of claim 19, wherein the at least one conductor includes at least one relay having a first end extending through one of the surfaces and a second end coupled in communication with the central rod.

21. A method of fusing bones, the method including:
inserting an implant body through a surgical opening into a space between the two bones;
sensing an electrical field from nerves adjacent the implant body with at least one conductor extending through a portion of the body; and
adjusting a position of the implant body based on the sensing of the electrical field.

22. A method of claim 21, wherein sensing the electrical field includes conducting a signal through relays to a draw rod.

23. A method of claim 22, further comprising connecting the draw rod to a computer system and collecting sensing data from the implant and conductor and displaying the sensing data on a graphical-user interface.
